(19) United States
(12) Patent Application Publication
(10) Pub. No.: US 2008/0113033 A1
(43) Pub. Date: May 15, 2008

(54) TOPICAL TREATMENT AND MANUFACTURING METHOD

(75) Inventors: Teri L. Koelzer, Corning, KS (US); William J. Koelzer, Corning, KS (US)

Correspondence Address: MARK BROWN, 4700 BELLEVIEW SUITE 210, KANSAS CITY, MO, 64112, US

(73) Assignee: HRK, Inc.

(21) Appl. No.: 11/939,245

(22) Filed: Nov. 13, 2007

Related U.S. Application Data
(60) Provisional application No. 60/858,536, filed on Nov. 13, 2006.

(51) Int. Cl. A61K 35/64; A61P 17/00; A61P 17/06

(52) U.S. Cl. 424/539

(57) ABSTRACT

A topical treatment for psoriasis, eczema and other disorders and conditions includes beeswax, coconut oil, shea butter and cocoa butter. Optional ingredients can also be included. A method of manufacturing the composition includes the steps of preheating and liquifying the beeswax, adding other ingredients, slowly stirring the resulting composition at an elevated temperature, and formulating finished treatment products, such as bars and lotions.

This application claims priority in U.S. Provisional Patent Application No. 60/858,536, filed Nov. 13, 2007, which incorporated herein by reference.

### BACKGROUND OF THE INVENTION

1. Field of the Invention

The present invention relates generally to topical treatments, and in particular, to a treatment for psoriasis, eczema and similar conditions using beeswax and a method for manufacturing a beeswax composition.

2. Description of the Related Art

Various topical treatments have been formulated and used for psoriasis, eczema and other skin disorders. For example, the Jaros et al. U.S. Pat. No. 6,316,001; No. 6,379,715 and No. 6,406,707 disclose methods for treating psoriasis. Beeswax has been disclosed in connection with the treatment of psoriasis. For example, the Chiba U.S. Pat. No. 4,325,965 discloses an agent for preventing or treating psoriasis. However, previous treatments for psoriasis, eczema and other skin disorders have lacked the advantages and features of the present invention.

### SUMMARY OF THE INVENTION

In the practice of an aspect of the present invention, a topical treatment is provided for psoriasis, eczema and other skin disorders. The treatment includes beeswax, coconut oil, shea butter and cocoa butter. In the practice of another aspect of the present invention, a method of manufacturing a treatment composition includes the steps of melting or softening beeswax, adding other ingredients, stirring the ingredients at an elevated temperature and pouring the mixed liquid into molds for casting bars for personal use.

### BRIEF DESCRIPTION OF THE DRAWING

FIG. 1 is a flow chart showing a method of manufacturing a treatment composition embodying an aspect of the present invention.

### DETAILED DESCRIPTION OF THE PREFERRED EMBODIMENTS

### 1. Introduction and Environment

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

### II. Topical Treatment

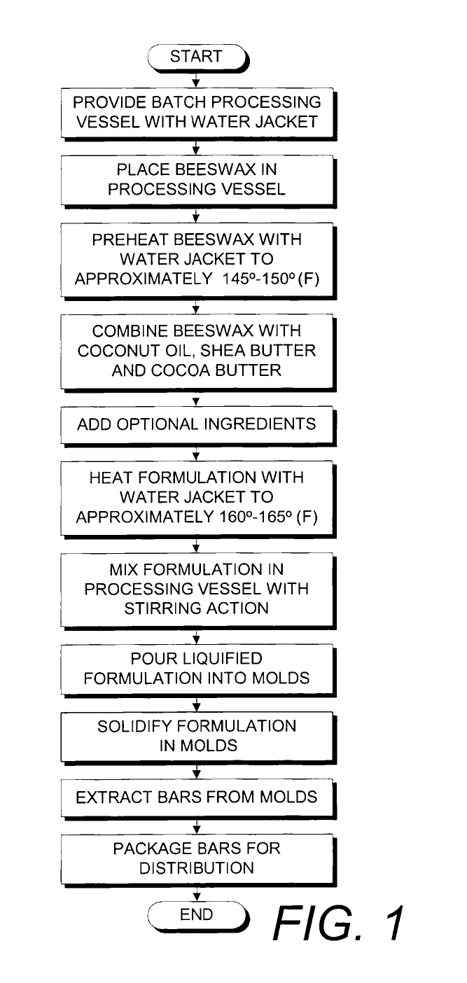

Referring to the drawing (FIG. 1) in more detail, a method for formulating a topical treatment, which includes the step of providing a vessel, such as a kettle with a water jacket. Such vessels are commonly used for heating liquids and other substances by heating the water in the water jacket. Beeswax is added to the vessel and heated to a temperature of approximately 145° to 150° F., which is sufficient to liquify the beeswax. Coconut oil, shea butter and cocoa butter are added to the liquefied beeswax.

The composition can comprise, for example, 10 parts beeswax, 10 parts coconut oil, 6.67 parts shea butter and 4 parts coconut butter. Such ingredients and their respective proportions can be modified as appropriate for various applications of the composition comprising the present invention.

The composition is heated to approximately 160° to 165° F. and maintained at approximately this temperature for approximately two to four hours by maintaining the heat of the water in the water jacket. Appropriate temperature control mechanisms, thermostats, sensors and other control equipment can be utilized for automating the methodology of the present invention. The heated composition can be gently stirred to thoroughly mix the ingredients at an elevated temperature. For example, motorized mixing equipment is commonly used for mixing dairy products in bulk tanks with motor-driven paddles operating at relatively low speeds. Such equipment can be adapted to implement the method of the present invention. The resulting composition can be poured into other vessels, such as pitchers, for pouring into suitable molds, or it can be distributed directly to the molds from the water-jacketed vessel. The composition solidifies into bars for personal use in the molds, which can be cast in any suitable shape for decorative or functional purposes. Alternatively, the composition can be emulsified, formulated into a lotion with suitable viscosity or formulated into any other suitable configuration. Without limitation, additional bee-derived ingredients can optionally include royal jelly, which is rich in nutrients such as protein, and propolis, which forms a protective barrier in bee hives.

### III. Treatment Method and Indications

The composition is particularly effective as a topical treatment for skin disorders, such as psoriasis and eczema. The coconut oil, shea butter and cocoa butter ingredients provide deep-penetrating moisturization, while the beeswax forms a protective surface barrier resisting wash-off and depletion of the moisturizing ingredients. The optional royal jelly ingredient provides protein-rich nutrients, which can promote healing and re-epithelialization. Propolis provides a surface barrier, which can block bacteria infiltration and help ward off infections. In addition to the aforementioned treatment applications of the composition, it can be utilized as a moisturizer and treatment for dry, cracked skin. Still further, it can provide a protective barrier against poison ivy and other irritants.

It will be appreciated that the composition can be utilized for other applications and topical therapeutic treatment methods. Moreover, additional and/or substitute ingredients can be included in order to achieve the desired effects. For example, alternative moisturizers, nutrients, surface barriers, fragrances, carriers and other substances can be utilized.

It is to be understood that the invention can be embodied in various forms, and is not to be limited to the examples discussed above. Other components and configurations can be utilized in the practice of the present invention.

What is claimed and desired to be secured by Letters Patent is as follows:

1. A topical treatment, which comprises:
beeswax;
coconut oil;
shea butter; and
cocoa butter.

2. The treatment according to claim 1, which comprises a composition of:
approximately 8-12 parts beeswax;
approximately 8-12 parts coconut oil approximately 5-8 parts shea butter; and
approximately 3-5 parts coconut butter.

3. The treatment according to claim 2 wherein said composition is formed in a bar.

4. A method of formulating a treatment, which comprises the steps of:
providing a batch processing vessel with a water jacket;
placing beeswax in the processing vessel;
preheating the beeswax with the water jacket to approximately 145°-150° (F.);
combining said beeswax with coconut oil, shea butter and coconut butter;
heating the formulation with the water jacket to approximately 160°-165° (F.);
mixing the formulation in the processing vessel with a stirring action;
pouring the liquefied formulation into a mold;
solidifying the formulation in said mold into a bar; and
extracting a bar from said mold.

5. The method according to claim 4, which includes the additional steps of:
providing approximately 8-12 parts beeswax;
providing approximately 8-12 parts coconut oil;
providing approximately 5-8 parts shea butter; and
providing approximately 3-5 parts coconut butter.

6. A. method of treating psoriasis, eczema and other skin disorders, which comprises the steps of:
applying a treatment composition comprising beeswax, coconut oil, shea butter and coconut butter.

7. The method according to claim 6, which includes the additional steps of:
providing approximately 8-12 parts beeswax;
providing approximately 8-12 parts coconut oil;
providing approximately 5-8 parts shea butter; and
providing approximately 3-5 parts coconut butter.
